(19) United States
(12) Patent Application Publication
(10) Pub. No.: US 2006/0178352 A1
(43) Pub. Date: Aug. 10, 2006

(54) Use of androgens to reduce the likelihood of acquiring or to treat skin aging

(75) Inventors: Mohamed El-Alfy, Sainte-Foy (CA); Fernand Labrie, Sainte-Foy (CA); Lamia Azzi, Sainte Foy (CA)

Correspondence Address: OSTROLENK FABER GERB & SOFFEN, 1180 AVENUE OF THE AMERICAS, NEW YORK, NY, 100368403, US

(73) Assignee: Endorecherche, Inc.

(21) Appl. No.: 11/264,704

(22) Filed: Nov. 1, 2005

Related U.S. Application Data
(60) Provisional application No. 60/624,112, filed on Nov. 1, 2004.

(51) Int. Cl. A61K 31/57

(52) U.S. Cl. 514/177; 514/178

(57) ABSTRACT

Novel methods of treating or reducing the likelihood of acquiring skin diseases due to age-related androgen deficiency, particularly skin atrophy, loss of collagen, loss of elastic fibers, loss of connective tissue, cellulite, and formation of wrinkles, in susceptible warm-blooded animals including humans involving administration of an androgen or/and a sex steroid precursor. Pharmaceutical compositions for delivery of active ingredient(s) useful to the invention are also disclosed.

### CROSS-REFERENCE TO RELATED APPLICATION(S)

The present application claims priority under 35 U.S.C. §119 of Provisional Application Serial No. 60/624,112 filed Nov. 1, 2004.

### FIELD OF THE INVENTION

The present invention relates to a method for treating or reducing the likelihood of acquiring skin diseases due to age-related androgen deficiency, comprising administering an effective amount of androgens or sex steroid precursors and prodrugs thereof to susceptible warm-blooded animals, including humans. In particular, the invention includes administering androgenic compounds or a precursor of sex steroids selected from the group consisting of dehydroepiandrosterone (DHEA), dehydroepiandrosterone sulfate (DHEA-S), and androst-5-ene-3β,17β-diol (5-diol), androstenedione, testosterone, DHT, androstanedione and androstane-3α, 17-β diol or prodrug, compounds transformed to any of these in vivo. The invention also relates to topical or oral pharmaceutical compositions for practicing the foregoing method.

### BACKGROUND OF THE RELATED ART

The almost exclusive focus on the role of ovarian estrogens in women has removed the attention from the dramatic 70% fall in circulating DHEA which already occurs between the ages of 20 to 30 and 50 to 60 years (Migeon et al., 1957, JCEM, 17: 1051-1062; Vermeulen and Verdonck, 1976, JCEM, 42: 247-253; Vermeulen et al., 1982, JCEM, 54: 187-191; Orentreich et al., 1984, JCEM 59: 551-555; Belanger et al., 1994, JCEM, 79: 1086-1090; Labrie et al., 1997, Labrie et al., 1997, JCEM, 82: 2396-2402). Since DHEA is transformed to both androgens and estrogens in peripheral tissues, such a fall in serum DHEA and DHEA-S shows why women at menopause are not only lacking estrogens but have also been progressively deprived of androgens for a few years.

The marked reduction in the formation of DHEA-S by the adrenals during aging (Migeon et al., 1957, JCEM, 17: 1051-1062; Vermeulen and Verdonck, 1976, JCEM, 42: 247-253; Vermeulen et al., 1982, JCEM, 54: 187-191; Orentreich et al., 1984, JCEM 59: 551-555; Bélanger et al., 1994, JCEM, 79: 1086-1090) results in a dramatic fall in the tissue-specific formation of androgens and estrogens in peripheral target tissues, a situation that has been proposed to be associated with age-related diseases including insulin resistance (Coleman et al., 1982, Diabetes, 31: 830-833; Schriock et al., 1988, JCEM, 66: 1329-1331) and obesity (Nestler et al., 1988, JCEM, 66:57-61; MacEwen and Kurzman, 1988, J Nutrit, 121: S51-S55; Tchernof et al., 1995, Tchernof et al., 1995, Diabetes Care, 18: 292-299). Moreover, much attention has been given to the benefits of DHEA administered to postmenopausal women, especially on the bone, sebaceous glands, vagina and well being after oral (Morales et al., 1994, JCEM, 78: 1360-1367; Baulieu et al., 2000, Proc Natl Acad Sci USA, 87: 4279-4284) as well as percutaneous (Diamond et al., 1996, J Endocrinol, 150: S43-S50; Labrie et al., 1997, JCEM, 82: 3498-3505) administration of the precursor steroid.

The data showing the presence of relatively high levels of androgens in normal women strongly suggest that the androgens play a major but so-far unrecognized physiological role in women. In fact, the 44.5% fall which occurs in serum DHEA from 20-30 years of age to the age of 40 to 50 years in women could well explain the early bone loss and the increased FSH/LH ratio which precede the detectable decrease in ovarian steroidogenesis in perimenopausal women. In fact, serum FSH increases in premenopausal women even before serum E₂ shows a decrease (Grodin et al., 1973, JCEM, 36: 207-214). On the other hand, age-related bone loss has been reported to begin during the fourth decade while changes in bone turnover have also been found before menopause (Riggs et al., 1981, J Clin Invest, 67: 328-335; Mazess et al., 1982, Clinic Orthop, 165: 239-252; Johnston et al., 1985, JCEM, 61: 905-911). In agreement with these findings, bone density was lower at all sites examined in women classified as perimenopausal compared to premenopausal (Steinberg et al., 1989, JCEM, 69: 533-539).

Until recently, due to assay difficulties, only a limited number of circulating adrenal and gonadal steroids had been measured during advancing age, especially in women where the impact of androgens and estrogens of adrenal origin is of particular importance (Labrie et al., 1991, Mol Cell Endocrinol, 78: C113-C118). It is thus quite remarkable that most of the important decline in circulating DHEA, DHEA-S, androst-5-ene-diol-3β, 17β-diol (5-diol), 5-diol-G, androstenedione (4-dione) as well as the conjugated metabolites of androgens, namely androsterone-glucuronide (ADT-G) and androstane-3α, 17β-diol glucuronide (3α-diol-G), occurs between the age ranges of 20-30 and 50-60 years while relatively small changes occur after the age of 60 years. It is thus important to notice that in the 50-60 year-old age group, serum DHEA has already decreased by 70% from the 20-30 year-old peak values (Labrie et al., 1997, JCEM, 82: 2396-2402). Such data suggest that androgenic hormone replacement therapy should start early, taking into account the marked decrease in androgens which occurs relatively early during aging in women.

As well demonstrated in our previous studies, supplementation with physiological amounts of exogeneous DHEA permits the biosynthesis of androgens and estrogens only in the appropriate target tissues which contain the required and tissue-specific steroidogenic enzymes. The active androgens and estrogens thus synthesized in specific peripheral tissues exert their action in the same cells that are responsible for their formation and very little leakage of the active steroids occurs into the circulation. In fact, as mentioned above, the most striking effects of DHEA administration are seen on the circulating levels of the glucuronide derivatives of the metabolites of DHT, namely ADT-G and 3α-diol-G, these metabolites being produced locally in the peripheral intracrine tissues which possess the appropriate steroidogenic enzymes to synthesize DHT from the adrenal precursors DHEA and DHEA-S (Labrie et al., 1991, Mol Cell Endocrinol, 78: C113-C118; Labrie et al. 1996, J Endocrinol, 150: S107-S118). This local biosynthesis and action of androgens in target tissues eliminates the exposure of other tissues to active androgens and thus minimizes the risks of undesirable masculinizing or other androgen-related side effects. The same applies to estrogens although we feel that a reliable parameter of total estrogen secretion (comparable to the glucuronides for androgens) is not yet available.

DHEA has been shown to have important effects on the skin of aged individuals, the most salient of which is an increase in sebum production (Labrie et al., 1997, JCEM, 82: 3498-3505). This has been shown in a number of studies performed in women, particularly those >70 years old who are physiologically hyposeborrheic and thus found an improvement of their skin with DHEA administration. The DHEA-induced increase in sebum production observed in our study is probably due to the fact that the sebaceous glands contain all the steroidogenic enzymes necessary to catalyze the transformation of DHEA into the androgen DHT, and that this androgen is the main stimulator of sebaceous gland activity (Labrie et al., 2000, Horm Res, 54: 218-219; Labrie et al., 2003, End Rev, 24: 152-182).

Apart from sebum production, other beneficial effects of DHEA on the skin have been noticed. To date, evaluation of the dermatological aspects of DHEA administration have only been performed with some details in one study in which male and female subjects between the ages of 60 and 79 years were orally administered 50 mg of DHEA, once daily for 1 year. In that study, (Baulieu et al., 2000, Proc Natl Acad Sci USA, 97: 4279-4284) were evaluated skin hydratation, skin pigmentation and skin thickness. Skin surface hydratation significantly increased for the whole DHEA-treated population examined after 12 months of treatment. Skin surface hydratation is considered a real benefit for the skin, especially in aged individuals since in these subjects the dryness makes the skin rough. DHEA also significantly decreased facial skin pigmentation (yellowness) for the whole population. This decrease was more pronounced in women >70 years who are more concerned by age-related pigment changes. The two other components of skin colour remained stable during the duration of the study (i.e. lightness and redness).

In U.S. Pat. No. 5,843,932, a method for treating skin atrophy or of inhibiting loss of collagen or connective tissue by administration of DHEA, DHEA-S or compounds converted in vivo to either of the foregoing is disclosed.

### SUMMARY OF THE INVENTION

It is an object of the present invention to provide effective methods of treatment of skin diseases due to age-related androgen deficiency.

It is another object of the present invention to provide effective methods of treatment of skin diseases due to age-related sex-steroid precursor deficiency.

It is another object to provide methods of reducing the risk of acquiring the above problems.

In one embodiment, the invention pertains to a method of treating or reducing the risk of acquiring skin atrophy, comprising administering to a patient in need of such treatment or reduction of risk an effective amount of androgens or prodrugs thereof.

In another embodiment, the invention pertains to a method of treating or reducing the risk of acquiring loss of collagen, comprising administering to patient in need of such treatment or reduction of risk an effective amount of androgens or prodrugs of thereof.

In another embodiment, the invention pertains to a method of treating or reducing the risk of acquiring loss of elastic fibers, administering to patient in need of such treatment or reduction of risk an effective amount of androgens or prodrugs of thereof.

In another embodiment, the invention pertains to a method of treating or reducing the risk of acquiring loss of elastic fibers, administering to patient in need of such treatment or reduction of risk an effective amount of sex steroid precursor or prodrugs of thereof.

In another embodiment, the invention pertains to a method of treating or reducing the risk of acquiring loss of connective tissue, comprising administering to patient in need of such treatment or reduction of risk an effective amount of androgens or prodrugs of thereof.

In another embodiment, the invention pertains to a method of treating or reducing the risk of acquiring cellulite, comprising administering to patient in need of such treatment or reduction of risk an effective amount of androgens or prodrugs thereof.

In another embodiment, the invention pertains to a method of treating or reducing the risk of acquiring formation of wrinkles, comprising administering to patient in need of such treatment or reduction of risk an effective amount of androgens or prodrugs of thereof.

In another embodiment, the invention pertains to a method of treating or reducing the risk of acquiring cellulite, comprising increasing levels of a sex steroid precursor selected from the group consisting of dehydroepiandrosterone (DHEA), dehydroepiandrosterone-sulfate (DHEA-S) and androst-5-ene-3β,17β-diol (5-diol), in a subject or patient in need of said treatment or said steroid precursor.

In another embodiment, the invention pertains to a method of treating or reducing the risk of acquiring formation of wrinkles, comprising increasing levels of a sex steroid precursor selected from the group consisting of dehydroepiandrosterone (DHEA), dehydroepiandrosterone-sulfate (DHEA-S) and androst-5-ene-3β,17β-diol (5-diol), in a subject or patient in need of said treatment or said steroid precursor.

In another embodiment, the invention pertains to a method of treating or reducing the risk of acquiring loss of elastic fibers, comprising increasing levels of a sex steroid precursor selected from the group consisting of dehydroepiandrosterone (DHEA), dehydroepiandrosterone-sulfate (DHEA-S) and androst-5-ene-3β,17β-diol (5-diol), in a subject or patient in need of said treatment or said steroid precursor.

In another embodiment, the invention pertains to a method of treating or reducing the risk of acquiring skin atrophy, comprising administering androstenedione, androstanedione, testosterone, dihydrotestosterone, 5α-androstane-3α,17β-diol or compounds transformed into these, in a subject or patient in need of said treatment.

In another embodiment, the invention pertains to a method of treating or reducing the risk of acquiring loss of collagen, comprising administering androstenedione, androstanedione, testosterone, dihydrotestosterone, 5α-androstane-3α,17β-diol or compounds transformed into these, in a subject or patient in need of said treatment.

In another embodiment, the invention pertains to a method of treating or reducing the risk of loss of elastic fibers, comprising administering androstenedione, androstanedione, testosterone, dihydrotestosterone, 5α-androstane-3α,17β-diol or compounds transformed into these, in a subject or patient in need of said treatment.

In another embodiment, the invention pertains to a method of treating or reducing the risk of loss of connective tissue, comprising administering androstenedione, androstanedione, testosterone, dihydrotestosterone, 5α-androstane-3α,17β-diol or compounds transformed into these, in a subject or patient in need of said treatment.

In another embodiment, the invention pertains to a method of treating or reducing the risk of acquiring wrinkles, comprising administering androstenedione, androstanedione, testosterone, dihydrotestosterone, 5α-androstane-3α,17β-diol or compounds transformed into these, in a subject or patient in need of said treatment.

In another embodiment, the invention pertains to a method of treating or reducing the risk of acquiring cellulite, comprising administering androstenedione, androstanedione, testosterone, dihydrotestosterone, 5α-androstane-3α,17β-diol or compounds transformed into these, in a subject or patient in need of said treatment.

In another aspect, the invention provides topical pharmaceutical compositions containing the androgens together with pharmaceutically acceptable diluents or carriers.

In one embodiment, the precursor is DHEA.

In another embodiment, the androgen is testosterone or its derivatives.

As used herein, an androgen is a compound (or one of its metabolites) having a Ki value for the human androgen receptor of less than about 2×10⁻⁸M and an androgen receptor-mediated inhibitory effect on the growth of human breast cancer ZR-75-1 cells which reaches half-maximal value at a concentration below 10 nanomoles per liter or a compound (or one of its metabolites) which positively responds to the screening method described in the US provisional application entitled “Method for determination of anabolic activity” filed on Aug. 30, 2004, application Ser. No. 60/606,174.

A patient in need of treatment or of reducing the risk of onset of a given disease is one who has either been diagnosed with such disease or one who is susceptible to acquiring such disease.

Except where otherwise stated, the preferred dosage of the active compounds (concentrations and modes of administration) of the invention is identical for both therapeutic and prophylactic purposes. The dosage for each active component discussed herein is the same regardless of the disease being treated (or of the disease whose likelihood of onset is being reduced).

Except when otherwise noted or where apparent from context, dosages herein refer to weight of active compounds unaffected by pharmaceutical excipients, diluents, carriers or other ingredients, although such additional ingredients are desirably included, as shown in the examples herein. Any dosage form, specially topical formulations (gel, lotion, cream, ointment or the like) commonly used in the pharmaceutical industry is appropriate for use herein, and the terms “excipient”, “diluent”, or “carrier” include such nonactive ingredients as are typically included, together with active ingredients in such dosage forms in the industry. For example, typical cream, penetrating agent, preservatives, or the like may be included for topical formulations.

All of the active ingredients used in any of the therapies discussed herein may be formulated in pharmaceutical compositions which also include one or more of the other active ingredients. Alternatively, they may each be administered separately but sufficiently simultaneous in time so that a patient eventually has elevated blood levels or otherwise enjoys the benefits of each of the active ingredients (or strategies) simultaneously. In some preferred embodiments of the invention, for example, one or more active ingredients are to be formulated in a single pharmaceutical composition.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the comparison between male and female mouse dorsal skin. (A) Paraffin sections of mouse dorsal skin stained with hematoxylin and eosin. a) In the intact male, all hair follicles are in the telogen phase and located in the dermis, which is bordered by a thin layer of hypodermis. b) In the intact female, all hair follicles are also in the telogen phase, while the hypodermis is thicker in comparison to the male, and the dermis is narrower. c) After GDX, the hair follicles of the male are in late anagen. d) In the GDX female, all the hair follicles are in anagen. Epidermis (E), dermis (D), hypodermis (H), panniculus carnosus (PC) and hair follicles (HF). Scale bar=100 μm. (B) Comparison between male and female total skin thickness as well as the thickness of each skin layer (epidermis, dermis and hypodermis) in intact and GDX animals and in GDX animals treated with DHT, E₂ or DHEA. Values are presented as means±SEM. *p<0.05 vs. GDX male control; ++p<0.01 vs. GDX female control (Duncan-Kramer multiple-range-test).

FIG. 2 shows the expression of androgen receptor (AR) in the epidermis of mouse dorsal skin. AR was found to be exclusively localized in the nuclei. a) in the intact male, most of epidermal nuclei are labeled. b) in the intact female, most of epidermal nuclei are labeled but the labeling intensity is less than in the male. Three weeks after GDX, no labeling could be detected in the GDX males (c) nor in the GDX females (d). When GDX male (e) and female (f) mice were treated with DHT, strong AR labeling was observed in most of the nuclei of epidermal cells. When GDX males (g) and females (h) received E₂, weak AR labeling was detected in some nuclei. Similar to DHT treatment, when GDX males (i) and females (j) received DHEA, a strong labeling was detected in most of the epidermal nuclei. Scale bar=20 μm.

FIG. 3 and FIG. 4 show a comparison of the face skin treated with DHEA (right side of the face) or untreated (left side of the face). Three-hundred microliters (0.3 ml) of the emulsion containing DHEA were applied on the forehead and on the right side of the face during 13 weeks.

### DETAILED DESCRIPTION OF THE INVENTION

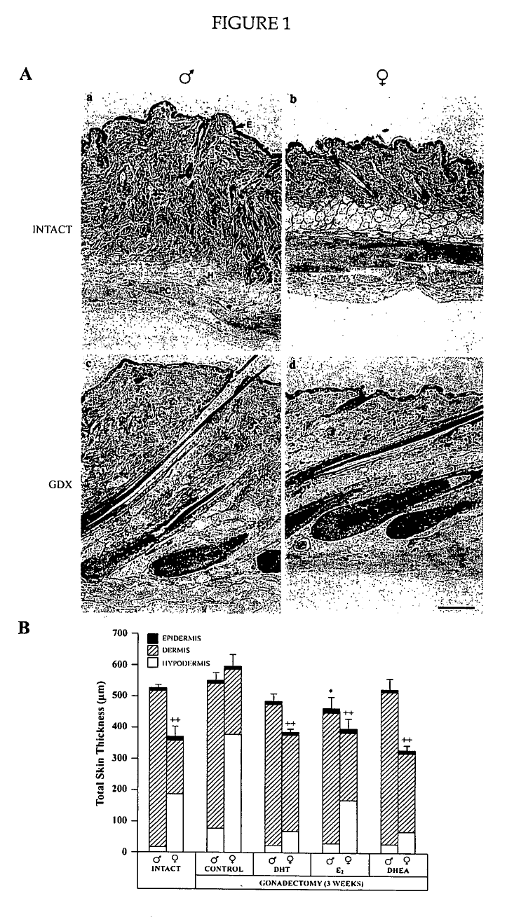

The present invention shows that a major effect of androgens was seen on dermal thickness. In fact, collagen and elastic fibers are known to be the main component of the dermis that provides a major support for skin resistance, including a possible role in the formation of wrinkles. An increase in dermal thickness was observed after DHT and DHEA treatment in GDX females, (FIG. 1) while, under all experimental conditions, the dermis is thicker in males, thus possibly explaining the lack of effect of androgens in the male during a 3-week treatment period. Striking gender differences are seen in the hypodermis of intact animals, thus providing further similarities with human skin (Hattori et al, 1993) and suggesting that DHEA and androgens could be beneficial for the reducing the risk of acquiring or treating cellulite.

Using the mouse as a model, the present invention clearly establishes morphological differences between males and females in the different mouse skin layers and appendages. Moreover, the specific and differential role of androgens and estrogens at different sites has been identified while providing evidence for an action of DHEA mediated by androgens in the dermis and estrogens in the epidermis.

Active ingredient for topical application is preferably present at from 0.05% to 20% by weight relative to the total weight of the pharmaceutical composition, more preferably between 0.1 and 10% DHEA or 5-diol and between 0.1% and 3% for an androgen. Alternatively, the active ingredient may be placed into a transdermal patch having structures known in the art, for example, structures such as those set forth in E.P. Patent No. 0279982.

When formulated as an ointment, lotion, gel or cream or the like, the active compound is admixed with a suitable carrier which is compatible with human skin or mucosa. Suitable carriers are known in the art and include but are not limited to Klucel H F and Glaxal base. Some are commercially available, e.g., Glaxal base available from Glaxal Canada Limited Company. Other suitable vehicles can be found in Koller and Buri, S. T. P. Pharma 3(2), 115-124, 1987. The carrier is preferably one in which the active ingredient(s) is (are) soluble at ambient temperature at the concentration of active ingredient that is used. The carrier should have sufficient viscosity to maintain the steroid on a localized area of skin or mucosa to which the composition has been applied, without running or evaporating for a time period sufficient to permit substantial penetration of the precursor or androgen through the localized area of skin or mucosa to cause a desirable clinical effect. The carrier is typically a mixture of several components, e.g. pharmaceutically acceptable solvents and a thickening agent. A mixture of organic and inorganic solvents can aid hydrophylic and lipophylic solubility, e.g. water and an alcohol such as ethanol and propylene glycol.

Preferred sex steroid precursors are dehydroepiandrosterone (DHEA) (available from Diosynth Inc., Chicago, Ill., USA), 5-androsten-3β,17β-diol (available from Steraloids, Wilton, N.H. USA).

One other preferred sex steroid precursor is 4-androstene-3,17-dione, available from Sigma-Aldrich Canada Ltd, Oakvill, Ontario, Canada.

One preferred androgen of the invention is Stanolone (5α-androstane-17β-ol-3-one, DHT), available from Sigma-Aldrich Canada Ltd, Oakvill, Ontario, Canada.

Another preferred androgen is AndroGel a gel containing 1% of testosterone in alcohol, water, Carbopol 980 NF, isopropyl myristate and 0.1 M sodium hydroxide, and available from Solvay Pharma, Markham, Ontario, Canada

One preferred androgen for systemic action is Androderm, a patch containing 12,2 mg or 24.3 mg of testosterone, available from Laboratoires Paladin Inc., Montréal, Quebec, Canada.

Other esters of testosterone (testosterone undecanoate, available from Organon Canada Ltd., Scarborough, Ontario, Canada, under the name andriol, testosterone enanthate, available from Theramed Corporation, Mississauga, Ontario, Canada under the name Delatestryl, testosterone cypionate available from Pfizer Canada, Kirland, Canada under the name Depo-testosterone (cypionate) or from Sabex, 2002 Inc., Boucherville, Qc, Canada under the name testosterone cypionate injection USP) and derivatives [i.e. nandrolone (19-nor testosterone) and esters (nandrolone decanoate available from Organo Canada, Ltd Scarborough Ontario Canada under the name Deca-Durabolin), methyltestosterone available from sigma-Aldrich Canada Ltd., Oakvill, Ontario, Canada are also preferred.

It is also preferred that the androgen are 5α-androstane-3α, 17β-diol and 5α-androstane-3,17-dione, both available from Sigma-Aldrich Canada Ltd., Oakvill, Ontario, Canada.

It is preferred that the sex steroid precursor or the androgen is formulated as an alcoholic gel containing 1.0 to 10% of caprylic-capric triglyceride (Neobee M-5); 10 to 20% of hexylene glycol; 2.0 to 10% of diethyleneglycol monomethyl ether (Transutol); 2.0 to 10% of Cyclomethicone (Dow Corning 345); 1.0 to 2% of benzyl alcohol and 1.0 to 5.0% of hydroxypropylcellulose (Klucel HF).

It is also preferred that the sex steroid precursor or the androgen is formulated as a cream containing 2.0 to 4.0% of Laurylmethicone copolyol, 5.0 to 7.0% Cyclomethicone, 2.0 to 4.0% of mineral oil, 6.0 to 8.0% of Cetearyl isononoate, 0.5 to 1.5% of Eumulgin B₂, 0.01 to 0.1% of butylated hydroxytoluene, 49.0 to 60.0% of Propylene Glycol, 10 to 20% of water, 0.5 to 1.5% of Magnesium sulphate, 4.0 to 6.0% of ethanol and 0.1 to 3.0% of sex steroid precursor or androgen.

It is also preferred that the sex steroid precursor or the androgen is formulated as a cream containing 0.1 to 10% of sex steroid precursor or androgen, 10 to 25% of Emulsifying Wax, 5 to 20% of Light mineral oil, 0.5 to 2.0% of Benzyl alcohol, 20 to 40 % of Ethanol 95% and 20 to 40% of water.

It is also preferred that the sex steroid precursor or the androgen is formulated as a cream containing 0.1 to 10% of sex steroid precursor or androgen, 2 to 10% of Cetyl alcohol, 5 to 10% Cetyl Esters Wax, 0.25 to 0.5% of Phenylethyl alcohol, 5 to 10% of White Wax, 20 to 40% of water, 20 to 40% of Glycerol, 2.0 to 10.0% of Mineral oil, 1.0 to 5.0% of Sodium Lauryl sulfate, 3.0 to 6.0% of Glyceryl monostearate, 3.0 to 6.0% of Propyl glycol monostearate, and 1 to 5.0% of Methyl stearate.

It is also preferred that the sex steroid precursor or the androgen is formulated for oral administration as a capsule containing 10 to 50 mg of sex steroid precursor or androgen derivative.

The carrier may also include various additives commonly used in ointments and lotions and well known in the cosmetic and medical arts. For example, fragrances, antioxidants, perfumes, gelling agents, thickening agents such as carboxymethylcellulose, surfactants, stabilizers, emollients, coloring agents and other similar agents may be present.

Preferably, the attending clinician will, especially at the beginning of treatment, monitor an individual patient's overall response and serum levels of DHEA or androgen and, especially, monitor the patient's overall response to treatment, adjusting dosages as necessary where a given patients' metabolism or reaction to treatment is atypical.

Typical dose for topical administration of sex steroid precursor or androgen is 5 mg to 200 mg of active ingredient per day, per 50 kg of body weight, preferably 20 to 60 mg per day.

If oral administration is chosen, 10 to 100 mg active ingredient should be administered once daily per 50 kg of body weight.

### EXAMPLES OF EFFECTIVENESS OF THE PRESENT INVENTION

### Example 1

Materials and Methods

Aniamls and Treatments

Fifty six adult male and female C57BL6 mice 13-15 weeks of age were obtained from Harlan Laboratory (Indiana, USA). Mice were randomly distributed into 4 groups of 7 animals per group as follows: (1) intact control; (2) GDX control; (3) GDX+DHT (0.1 mg/mouse); (4) GDX+DHEA (6.25 mg/mouse). On day one of the study, bilateral GDX was performed as described (Castro, 1974 and Fleischman, 1981) in all animals except those of the first group which were sham-operated. Starting from the second day after GDX and for three weeks, DHEA is daily administrated orally as a suspension in 0.4% methylcellulose and 5% ethanol to the animals of the appropriate groups. Animals of intact and GDX control groups were treated with the vehicle alone during the same period. Six hours after the last treatment, all animals were sacrificed.

The oral doses of DHEA were selected based upon previous published studies (Labrie et al, 1996; Labrie et al, 2003b). Thus, the selected physiological doses completely reversed the GDX-induced atrophy of hormone-sensitive organs and led to organ weights similar to those found in intact animals. Since DHT is known to be poorly active by the oral route, it was injected subcutaneously. To determine the DHT dose, a preliminary dose-range study was performed (see supplementary online material, online Table S1).

Tissue Processing

After shaving the long hair, the dorsal skin was excised, flattened and immediately immersed in 10% buffered formalin. A sample was embedded in paraffin blocks from which 4 μm sections were cut and routinely stained. The other part of the skin was used for the whole mount technique as described (Badertscher J A, 1940, Stain Technol, 15: 29-30).

Skin Thickness Analysis

Under the light microscope, measurements were performed using the IMAGE-PRO PLUS (Media Cybernetics, USA). Twenty-five readings were scored from each skin layer of each animal. The epidermal thickness was measured from stratum basale to stratum granulosum (excluding stratum corneum), whereas the dermal thickness was the distance between the epidermis and the hypodermis. Finally, the hypodermal thickness was measured as the distance between the dermis and the panniculus carnosus.

Immunohistochemistry

Paraffin sections were deparaffinized and rehydrated. Endogenous peroxidase activity was eliminated by preincubation in 3% H₂O₂ in methanol for 30 min. A microwave retrieval technique using citrate buffer was applied (Tacha et Chen, 1994), and non-specific binding sites were neutralized with 10% goat serum. The sections were then incubated for 60 min at room temperature with mouse anti-Ki-67 antibody clone MIB-5 (1:60) (Dako Diagnostic, CA, USA) or for 90 min at room temperature with rabbit anti-androgen receptor (AR) antibody (1:300) (N-20; Santa Cruz Biotechnology, Inc., CA, USA). Zymed SP kit (San Francisco, Calif., USA) and Vectastain Elite ABC Kit (Vector Laboratories, Inc. Burlingame, Calif., USA) were used for AR, and Ki-67 antibodies, respectively. Under microscope monitoring, diaminobenzidine was used as the chromogen. For the evaluation of Ki-67, the labeling index of 400 cells was calculated from each animal.

Statistical Analysis

Data were expressed as means±S.E.M. The statistical significance was determined according to the multiple range test of Duncan-Kramer (Kramer CY, 1956, Biometrics, 12: 307-310).

Results

Morphological examination of dorsal skin of 16-to 18-week-old male and female mice reveals that gender differences in the global thickness of the skin and in the proportions of the different skin layers are clearly seen (FIGS. 1A and 1B). In fact, the major difference is that the dermis in the male is much thicker than in the female while the epidermis and hypodermis are thicker in the female, thus resulting in total skin which is 40% thicker in the male.

Epidermis

The epidermis of intact females is approximately 40% thicker than in males (p<0.01). Three weeks after GDX, the epidermal thickness of females decreased by 40% (p<0.01) while, a 13% increase was observed after DHEA treatment (p<0.05).

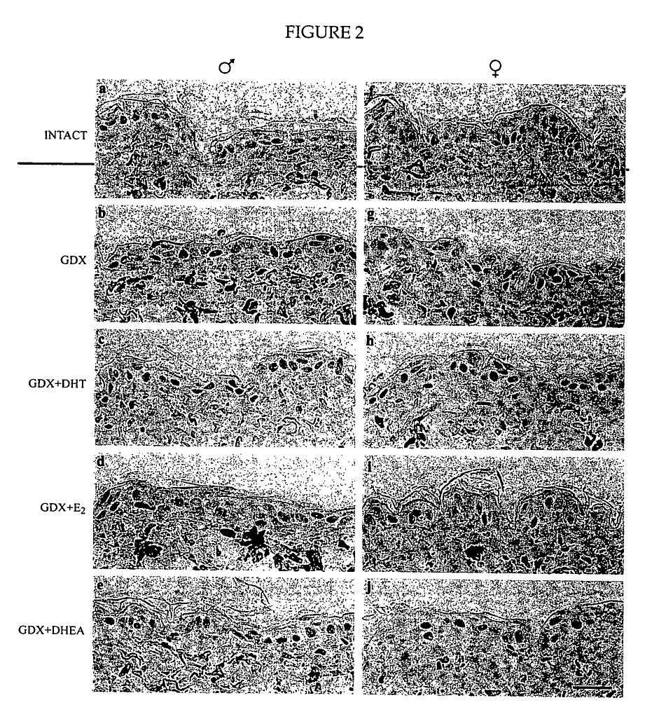

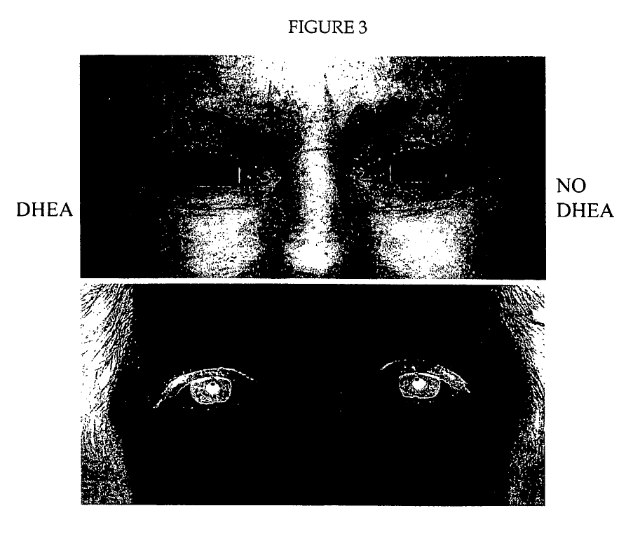

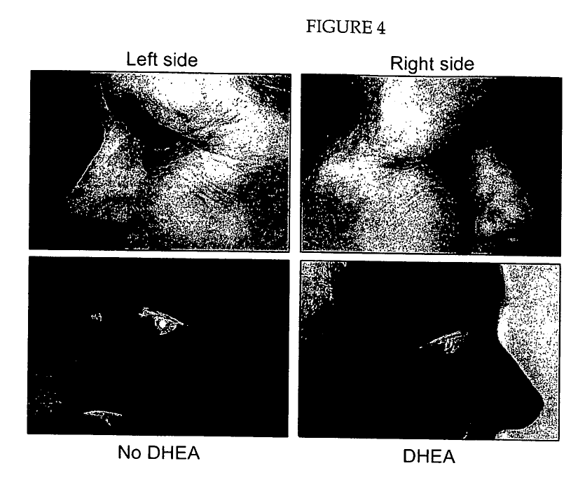

As indicated by the number of Ki-67-positive basal cells, cell proliferation was found to be higher in intact females (11.8±0.7 vs 9.3±0.4, p<0.05), thus indicating a gender difference. Furthermore, 3 weeks after GDX, the level of cell proliferation decreased in females by 27% (8.6±0.7, p<0.01), a value similar to that observed in intact males. AR immunostaining intensity was found to be slightly higher in intact males than females (FIGS. 2a, 2b). Three weeks after GDX, AR expression decreased in male epidermal cells to a level similar to that of females (FIGS. 2c, 2d). When GDX animals received DHT or DHEA, a strong AR expression was observed in both males and females (FIGS. 2e, 2i, 2f, 2j).

Dermis

In intact animals, the dermis was 190% thicker in the male compared to the female (p<0.01). After GDX, the female dermal thickness increased by 22% (p<0.05) while the 7% decrease in the male was not statistically significant. Only in GDX females, DHT and DHEA treatments significantly increased dermal thickness by 47% (p<0.01) and 19% (p<0.05), respectively.

Hypodermis

The hypodermis in the intact female was about 11-fold thicker than in the male (p<0.01). As seen in FIG. 1B, after GDX, hypodermal thickness increased in both male and female mice (p<0.01) while treatment with DHT, E₂ or DHEA markedly decreased hypodermal thickness in GDX animals of both sexes (p<0.01). These findings are a good indication that treatment with DHT, E₂ or DHEA is efficient for the treatment of cellulite.

TABLE I
Table 1. Gender differences in the relative proportions of the different skin layers.

| | Epidermis | | | | Dermis | | | | Hypodermis | | | |
|---|---|---|---|---|---|---|---|---|---|---|---|---|
| | ♂ | | ♀ | | ♂ | | ♀ | | ♂ | | ♀ | |
| | T (μm) | % | T (μm) | % | T (μm) | % | T (μm) | % | T (μm) | % | T (μm) | % |
| Intact | 9.4 ± 0.3 | 1.8 | 13.3 ± 0.4⁺⁺ | 3.6 | 500 ± 10.2 | 94.9 | 171 ± 13.3⁺ | 46 | 18 ± 1.5** | 3.3 | 187 ± 1.5⁺⁺ | 50.4 |
| GDX | 9.4 ± 0.2 | 1.7 | 9.3 ± 0.2 | 1.6 | 463 ± 21.3 | 84.1 | 209 ± 10.4 | 35 | 78 ± 5.8 | 14.2 | 378 ± 3.9 | 63.4 |
| GDX + DHT | 10 ± 0.2 | 2.1 | 9.6 ± 0.2 | 2.5 | 451 ± 24.1 | 93 | 307 ± 13.3⁺⁺ | 79.5 | 24 ± 1.9** | 4.9 | 70 ± 4.6⁺⁺ | 18 |
| GDX + DHEA | 8.9 ± 0.3 | 1.7 | 10.5 ± 0.3⁺ | 3.2 | 486 ± 28.7 | 92.8 | 249 ± 11.8⁺ | 75.8 | 29 ± 6.2** | 5.5 | 69 ± 4.5⁺⁺ | 21 |

Values are presented as means ± SEM.
**p < 0.01, experimental versus GDX male;
⁺⁺p < 0.01, experimental versus GDX female;
⁺p < 0.05, experimental versus GDX female (Duncan-Kramer multiple-range-test).
T: Thickness (μm);
%: Proportion of each skin layer.

### Example 2

### Protocol ERC-202

Study Design

This study is a randomized double-blind placebo-controlled trial of 15 subjects per arm. The study was divided into two phases, a screening period and a treatment period of 13 weeks.

Subjects and Treatment

After written informed consent was obtained, and women were found eligible, each subject was randomized to receive either a 0.0% (placebo), a 0.1%, a 0.3%, a 1% or a 2% DHEA emulsion twice a day in the morning and evening.

Daily, before breakfast, and after supper, for 13 weeks, subjects received 3.0 ml of one of the five emulsions.

All subjects were be instructed to apply the study treatment on the face (right side) and forehead, upper chest, back of hands, back of arms, external face of thighs and legs twice daily (in the morning between 06:00 and 09:30 h and in the evening between 18:00 and 21:30 h) during 13 weeks. The first application of the study treatment was carried out at the investigational site where instructions were provided to the subjects on how to apply the topical emulsion. Three-hundred microliters (0.3ml) of the emulsion were applied on the forehead and face (right side), 0.3 ml per back of arm and back of hand (0.3 ml×2), 0.3 ml on the upper chest, 0.6 ml per thigh (0.6 ml×2) and 0.3 ml per leg (0.3 ml×2) for a total dose of 3.0 ml of DHEA emulsion twice daily.

### Pharmaceutical Composition Examples

Set forth below, by way of example and not of limitation, are several topical pharmaceutical compositions utilizing preferred active sex steroid precursor DHEA, preferred androgens. The concentration of active ingredient may be varied over a wide range as discussed herein. The amounts and types of other ingredients that may be included are well known in the art.

### Example A

### Topical Cream

| | Weight % |
|---|---|
| Ingredient | (by weight of total composition) |
| DHEA | 1.0 |
| Emulsifying Wax, NF | 18.0 |
| Light mineral oil, NF | 12.0 |
| Benzyl alcohol | 1.0 |
| Ethanol 95% USP | 34.0 |
| Purified water, USP | 34.0 |

### Example B

### Topical Cream

### Cream 0.1% DHEA (Formulation No 0759-0435)

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 6.0 |
| Marcol 82 | 3.0 |
| Cetearyl isononoate (Cetiol SN) | 7.0 |
| Eumulgin B2 | 1.0 |
| B.H.T | 0.1 |
| Propylene Glycol | 58.4 |
| DHEA, GMP | 0.1 |
| Water, USP | 15.4 |
| Magnesium sulfate, 7 H2O | 1.0 |
| Absolute ethanol USP | 5.0 |
| Total: | 100.0 |

### Example C

### Topical Cream

### Cream 0.3% DHEA (Formulation No 0759-0435)

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 6.0 |
| Marcol 82 | 3.0 |
| Cetearyl isononoate (Cetiol SN) | 7.0 |
| Eumulgin B2 | 1.0 |
| B.H.T | 0.1 |
| Propylene Glycol | 58.4 |
| DHEA, GMP | 0.3 |
| Water, USP | 15.2 |
| Magnesium sulfate, 7 H2O | 1.0 |
| Absolute ethanol USP | 5.0 |
| Total: | 100.0 |

### Example D

### Topical Cream

### Cream 0.3% DHEA (Formulation No 0759-0484)

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 10.0 |
| Primol 352 | 3.0 |
| Cetearyl isononoate (Cetiol SN) | 7.0 |
| Eumulgin B2 | 1.0 |
| B.H.T | 0.1 |
| Propylene Glycol | 49.60 |
| DHEA, GMP | 0.3 |
| Water, USP | 20.00 |
| Magnesium sulfate, 7 H2O | 1.0 |
| Absolute ethanol USP | 5.0 |
| Total: | 100.0 |

### Example E

### Topical Cream

### Cream 1.0% DHEA (Formulation No 0759-0435)

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 6.0 |
| Marcol 82 | 3.0 |
| Cetearyl isononoate (Cetiol SN) | 7.0 |
| Eumulgin B2 | 1.0 |
| B.H.T | 0.1 |
| Propylene Glycol | 58.4 |
| DHEA, GMP | 1.0 |
| Water, USP | 14.5 |
| Magnesium sulfate, 7 H2O | 1.0 |
| Absolute ethanol USP | 5.0 |
| Total: | 100.0 |

### Example F

### Topical Cream

### Cream 1.5% DHEA (Formulation No 484-12-1.5%)

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 5.0 |
| Primol 352 | 3.0 |
| Cetearyl isononoate (Cetiol SN) | 7.0 |
| Eumulgin B2 | 1.0 |
| B.H.T | 0.090 |
| Propylene Glycol | 45.41 |
| ethanol USP | 14.0 |
| DHEA, GMP | 1.5 |
| Water, USP | 14.0 |
| Magnesium sulfate, 7 H2O | 0.65 |
| ethanol USP | 5.35 |
| Total: | 100.0 |

### Example G

### Topical Cream

### Cream 1.5% DHEA (Formulation No 435-3-1.5%)

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 6.0 |
| Primol 352 | 3.0 |
| Cetearyl isononoate (Cetiol SN) | 7.0 |
| Eumulgin B2 | 1.0 |
| B.H.T | 0.090 |
| Propylene Glycol | 58.41 |
| DHEA, GMP | 1.5 |
| Water, USP | 14.0 |
| Magnesium sulfate, 7 H2O | 1.0 |
| ethanol USP | 5.0 |
| Total: | 100.0 |

### Example H

### Topical Cream

### Cream 1.5% DHEA (Formulation No 47-1.5%)

| | Weight % |
|---|---|
| Ingredient | (by weight of total composition) |
| Laurylmethicone copolyol (DC | 3.0 |
| Emulsifier 10) | |
| Cyclometicone | 5.0 |
| Mineral oil | 3.0 |
| 2-ethylhexyl stearate | 10.0 |
| Cutina E24 | 1.0 |
| B.H.T | 0.090 |
| Propylene Glycol | 45.51 |
| ethanol USP | 10.0 |
| DHEA, GMP | 1.5 |
| Water, USP | 15.0 |
| Magnesium sulfate, 7 H2O | 0.65 |
| ethanol USP | 5.25 |
| Total: | 100.0 |

### Example I

### Topical Cream

### Cream 2.0% DHEA (Formulation No 0759-0435)

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 6.0 |
| Marcol 82 | 3.0 |
| Cetearyl isononoate (Cetiol SN) | 7.0 |
| Eumulgin B2 | 1.0 |
| B.H.T | 0.1 |
| Propylene Glycol | 58.4 |
| DHEA, GMP | 2.0 |
| Water, USP | 13.5 |
| Magnesium sulfate, 7 H2O | 1.0 |
| Absolute ethanol USP | 5.0 |
| Total: | 100.0 |

### Example J

### Topical Cream

### Cream 0.3% Testosterone

| | Weight % |
|---|---|
| Ingredient | (by weight of total composition) |
| Laurylmethicone copolyol (DC | 3.0 |
| Emulsifier 10) | |
| Cyclometicone (Mirasil CM5) | 10.0 |
| Primol 352 | 3.0 |
| Cetearyl isononoate (Cetiol SN) | 7.0 |
| Eumulgin B2 | 1.0 |
| B.H.T | 0.1 |
| Propylene Glycol | 49.60 |
| Testosterone GMP | 0.3 |
| Water, USP | 20.00 |
| Magnesium sulfate, 7 H2O | 1.0 |
| Absolute ethanol USP | 5.0 |
| Total: | 100.0 |

### Example K

### Topical Cream

### Cream 0.3% DHEA

| | Weight % |
|---|---|
| Ingredient | (by weight of total composition) |
| Cetyl alcohol | 8.0 |
| Mineral oil | 7.0 |
| Cetyl Esters Wax | 4.0 |
| White Wax | 6.0 |
| Glyceryl monostearate, | 4.0 |
| Propyl glycol monostearate | 2.0 |
| Methyl stearate | 1.0 |
| Phenylethyl alcohol | 0.5 |
| Glycerol | 44.70 |
| DHEA GMP | 0.30 |
| Water, USP | 21.50 |
| Sodium Lauryl Sulfate | 1.00 |
| Total: | 100.0 |

### Example L

### Topical Cream

### Cream 0.3% DHT

| | Weight % |
|---|---|
| Ingredient | (by weight of total composition) |
| Cetyl alcohol | 8.0 |
| Mineral oil | 7.0 |
| Cetyl Esters Wax | 4.0 |
| White Wax | 6.0 |
| Glyceryl monostearate, | 4.0 |
| Propyl glycol monostearate | 2.0 |
| Methyl stearate | 1.0 |
| Phenylethyl alcohol | 0.5 |
| Glycerol | 44.70 |
| DHT GMP | 0.30 |
| Water, USP | 21.50 |
| Sodium Lauryl Sulfate | 1.00 |
| Total: | 100.0 |

### Example M

### Topical Cream

### Cream 0.3% Testosterone

| | Weight % |
|---|---|
| Ingredient | (by weight of total composition) |
| Cetyl alcohol | 8.0 |
| Mineral oil | 7.0 |
| Cetyl Esters Wax | 4.0 |
| White Wax | 6.0 |
| Glyceryl monostearate, | 4.0 |
| Propyl glycol monostearate | 2.0 |
| Methyl stearate | 1.0 |
| Phenylethyl alcohol | 0.5 |
| Glycerol | 44.70 |
| Testosterone GMP | 0.30 |
| Water, USP | 21.50 |
| Sodium Lauryl Sulfate | 1.00 |
| Total: | 100.0 |

### Example N

### Topical Gel

### Gel 10.0% DHEA

| | Weight % |
|---|---|
| Ingredient | (by weight of total composition) |
| DHEA | 10.0 |
| Caprylic-capric Triglyceride | 5.0 |
| (Neobee M-5) | |
| Hexylene Glycol NF | 15.0 |
| Transcutol (diethyleneglycol | 5.0 |
| monomethyl ether) | |
| Benzyl alcohol | 2.0 |
| Cyclomethicone (Dow corning 345) | 5.0 |
| Ethanol (absolute) USP | 56.0 |
| Hydroxypropylcellulose (1500 cps) | 2.0 |
| (KLUCEL) | |
| Total: | 100.0 |

### Example O

### Topical Gel

### Gel 2.0% DHT

| | Weight % |
|---|---|
| Ingredient | (by weight of total composition) |
| Dihydrotestosterone | 2.0 |
| Caprylic-capric Triglyceride | 5.0 |
| (Neobee M-5) | |
| Hexylene Glycol NF | 15.0 |
| Transcutol (diethyleneglycol | 5.0 |
| monomethyl ether) | |
| Benzyl alcohol | 2.0 |
| Cyclomethicone (Dow corning 345) | 5.0 |
| Ethanol (absolute) USP | 64.0 |
| Hydroxypropylcellulose (1500 cps) | 2.0 |
| (KLUCEL) | |
| Total: | 100.0 |

### Example P

### Oral Capsules

### Capsule 50 mg DHEA

| | mg/capsule |
|---|---|
| Ingredient | Strength: 50 mg |
| DHEA (micronized) | 50.0 |
| Cellulose, Microcrystalline | 248.5 |
| (AVICEL 302) | |
| Magnesium Stearate | 1.50 |
| Total | 300.0 |

### Example Q

### Oral Capsules

### Capsule 25 mg DHEA

| | mg/capsule |
|---|---|
| Ingredient | Strength: 25 mg |
| DHEA (micronized) | 25.0 |
| (House standard) | |
| Cellulose, Microcrystalline | 273.5 |
| (AVICEL 302) (USP) | |
| Magnesium Stearate (NF) | 1.50 |
| Total | 300.0 |

The invention has been described in terms of preferred embodiments and examples, but is not limited thereby. Those of skill in the art will readily recognize the broader applicability and scope of the invention which is limited only by the patent claims herein.

What is claimed is:

1. A method for treating or reducing the likelihood of acquiring skin diseases due to age-related androgen deficiency, comprising administering to patient in need of such treatment or in need of reduction of risk an effective amount of androgens or prodrugs of thereof.

2. A method of claim 1 wherein these skin diseases are selected from the group consisting of skin atrophy, loss of collagen, loss of elastic fibers, loss of connective tissue, cellulite, and wrinkles.

3. The method of claim 1 wherein androgens or prodrugs of thereof are administered topically.

4. The method of claim 1 wherein androgens or prodrugs of thereof are administered orally.

5. The method of claim 1 wherein the androgen is selected from the group consisting of testosterone (4-androstene-17β-ol-3-one), dihydrotestosterone (5α-androstane-17β-ol-3-one), androstenedione (4-androstene-3,17-dione), androstanedione (5α-androstane-3,17-dione), 5α-androstane-3α, 17β-diol or precursors of these.

6. A method for treating or reducing the likelihood of acquiring wrinkles and cellulite or loss of elastic fibers comprising administering to patient in need of such treatment or in need of reduction of risk an effective amount of at least one androgen selected from the group consisting of testosterone (4-androstene-17β-ol-3-one), dihydrotestosterone (5α-androstane-17β-ol-3-one), androstenedione (4-androstene-3,17-dione), androstanedione (5α-androstane-3,17-dione), 5α-androstane-3α, 17β-diol or prodrugs of thereof.

7. A method for treating or reducing the likelihood of acquiring skin atrophy, loss of collagen, comprising administering to patient in need of such treatment or in need of reduction of risk an effective amount of at least one androgen selected from the group consisting of testosterone (4-androstene-17β-ol-3-one), dihydrotestosterone (5α-androstane-17β-ol-3-one), androstenedione (4-androstene-3,17-dione), androstanedione (5α-androstane-3,17-dione), 5α-androstane-3α, 17β-diol or prodrugs of thereof.

8. A method for treating or reducing the likelihood of acquiring wrinkles and cellulite or loss of elastic fibers comprising administering to patient in need of such treatment or in need of reduction of risk an effective amount of at least one sex steroid precursor selected from the group consisting of dehydroepiandrosterone, dehydroepiandrosterone-sulfate, 5-androstene-3β,17β-diol and prodrugs of thereof.

9. The method of claim 6 wherein sex steroid precursors are administered orally.

10. The method of claim 7 wherein sex steroid precursors are administered orally.

11. The method of claim 8 wherein sex steroid precursors are administered orally.

12. The method of claim 6 wherein sex steroid precursors are administered topically.

13. The method of claim 7 wherein androgens or prodrugs of thereof are administered topically.

14. The method of claim 8 wherein androgens or prodrugs of thereof are administered topically.
